(19) United States
(12) Patent Application Publication
(10) Pub. No.: US 2026/0284324 A1
(43) Pub. Date: Sep. 24, 2026

(54) SHIELDED NEEDLE UNIT WITH ROTATING NEEDLE

(71) Applicant: Novo Nordisk A/S, Bagsvaerd (DK)

(72) Inventor: Nicolai Michael Villadsen, Oelstykke (DK)

(21) Appl. No.: 19/476,221

(22) PCT Filed: May 16, 2024
(86) PCT No.: PCT/EP2024/063536

§ 371 Date: Oct. 17, 2025

(30) Foreign Application Priority Data
May 23, 2023 (EP) 23174841.9

(51) Int. Cl. A61M 5/32

(52) U.S. Cl. CPC A61M 5/3204

(57) ABSTRACT

A drug delivery assembly comprising a drug-filled cartridge, an actuatable drug-expelling mechanism, a needle hub with a needle protruding from the needle hub, and a shield in which the needle hub is fully or partly arranged, the shield being axially moveable relative to the needle hub between an extended position in which the shield axially covers the needle, and a retracted position in which the needle protrudes from the shield corresponding to a needle free length. The shield is mounted non-rotatable relative to the housing, the needle hub being rotatable relative to the shield and thus to the housing. The needle hub is rotated relative to the shield when the shield is moved from its retracted to its extended position, wherein rotational movement of the needle hub takes place after at least half of the needle free length has been covered by the shield.

The present invention generally relates to a needle unit as well as a drug delivery assembly comprising such a needle unit, the needle unit comprising a hollow hypodermic needle in-tended for subcutaneous introduction of a fluid drug formulation, the needle unit being mounted on or being adapted to be mounted on a drug delivery device by a user, the needle unit comprising an axially moveable needle shield designed to reduce the risk of accidental needle-stick injuries. A needle unit with a shielded needle is often termed a “safety needle”, especially if the shield is designed to lock in place after use. In the field of hypodermic needles sometimes the term “needle” is used and sometimes the broader term “cannula” may be used.

### BACKGROUND OF THE INVENTION

In the disclosure of the present invention reference is mostly made to hypodermic needles intended for use by a patient for subcutaneous administration of a fluid drug formulation, e.g. in the treatment of diabetes by delivery of insulin or a GLP-1 type drug, or in the treatment of growth disorders by delivery of growth hormone, however, these are only exemplary uses of the present invention.

To administer such drugs a great number of different drug delivery systems have been developed over the last 30 years. The drug delivery system is commonly in the form of a “pen” device (due to its form) comprising a cartridge containing the liquid drug to be injected. The pen device may be of the durable type adapted to receive a user-replaceable drug cartridge or it may be a “prefilled” disposable provided with a cartridge not intended to be replaced by the user. The pen device may be adapted to expel a single or a number of drug doses, the size of the dose being either user-settable or pre-set and thus not adjustable by the user. The expelling mechanism for driving the fluid drug out of the cartridge may be “manual” with the force applied to the pen device as the drug is being expelled. Alternatively the pen device may be “automatic” in which case a strained spring will provide the energy for the expelling mechanism. The spring may be strained during dose-setting, e.g. as in a FlexTouch® pen provided by Novo Nordisk A/S, or the spring may be provided pre-strained to the user with sufficient energy to expel one or more pre-set or user-set doses. As a further alternative the pen device may be motor-driven.

Common to all these different types of drug delivery devices is that an injection needle has to be provided allowing the drug to be administered subcutaneously. The needle may come pre-attached which is common for single-dose devices, however, it has also been proposed to provide multi-dose devices with a pre-mounted needle adapted for repeated use.

This said, for multi-dose pen devices the most common solution is to provide single-use needle units intended to be mounted by the user on the pen device prior to use and then discarded after use. The needle unit (also termed “needle module”) typically comprises a hub member adapted to be releasably mounted on the cartridge distal end, the hub member carrying a subcutaneous needle with a pointed distal end adapted to be inserted subcutaneously and a pointed proximal end adapted to be inserted through a needle-pierceable septum into the cartridge.

A problem presented by the handling and disposal of a pen needle assembly is the potential risk of being injured by any of the pointed ends of the needle. This is particular dangerous when following after the penetration of a patient's skin since the needle then may be contaminated and therefore capable of spreading diseases such as hepatitis and HIV.

Addressing this problem a great number of pen needle units have been developed where the patient end of the needle is concealed by a spring-loaded and telescopically movable shield during and after the injection, e.g. as disclosed in WO 01/91837 WO 03/066141, EP 1 289 587, EP 1 448 256 and U.S. Pat. No. 11,497,857.

When a shielded needle unit is provided the shield may also be used to release a spring-driven expelling mechanism. Such a needle unit may be formed integrally with a pen device typically for single-use or it may be provided as a user-mountable needle unit, e.g. as disclosed in WO 2018/215605.

Although it may be desirable to provide a pen needle unit with a shielding arrangement this must be balanced with the requirements for cost-effectiveness, safety and ease of handling and use.

Having regard to the above, it is an object of the present invention to provide a hypodermic needle unit of the shielded type which can be manufactured cost-effectively, which provides a high degree of safety, and which is both easy and safe to handle during operation and use. Such a needle unit may be provided as a stand-alone product adapted to be used in combination with one or more specific types of drug delivery devices, in combinations with a given drug delivery device, pre-mounted on a given drug delivery device or formed integrally with a drug delivery device.

### DISCLOSURE OF THE INVENTION

In the disclosure of the present invention, embodiments and aspects will be described which will address one or more of the above objects or which will address objects apparent from the below disclosure as well as from the description of exemplary embodiments.

The present invention is based on the realization that in a shielded needle unit in which the functionality, e.g. release of a mounting coupling, locking of the shield after use or controlling a container snap coupling, is controlled at least in part by the needle hub rotating relative to the drug delivery device per se, also the subcutaneous needle will rotate which may not be desirable.

Thus, in a first aspect of the invention a needle unit is provided, comprising a needle hub, a hollow needle mounted in the needle hub and having a pointed distal end protruding from the needle hub, the hollow needle defining a reference axis, and a shield in which the needle hub is fully or partly arranged. The shield is axially moveable relative to the needle hub between an extended position in which the shield axially covers the needle distal end, and a retracted position in which the needle distal end protrudes from the shield corresponding to a needle free length. The needle unit further comprises a coupling allowing the needle unit to be used in combination with a drug delivery device, and a linear-to-rotational movement converter mechanism causing the needle hub to be rotated relative to the shield when the shield is moved from its retracted to its extended position. The linear-to-rotational movement converter mechanism has an initial axial slack whereby rotational movement of the needle hub relative to the shield takes place after at least 25%, at least 50%, at least 75% or 100% of the needle free length has been covered by the shield.

By this arrangement it is prevented that the distal end of the pointed needle will rotate when the needle is fully inserted subcutaneously, the deeper structures of the skin being more susceptible to potential damage from a rotating needle than the more superficial skin structures.

It is to be noted that relative axial and rotational movement between the needle hub and the shield is defined. Correspondingly, when the needle unit is used in combination with a drug delivery device providing that the shield is moved axially relative to drug delivery device, then the needle hub is rotated relative to the drug delivery device. However, if the needle hub on its own is held rotationally fixed, then axial movement of the shield would result in rotation of the shield.

The linear-to-rotational movement converter mechanism may comprise one or more surfaces inclined relative to the reference axis and adapted to engage corresponding cooperating actuation surfaces providing that axial movement of the shield relative to the needle hub is converted to rotation of the needle hub relative to the shield. The inclined surface(s) may be arranged on either the needle hub or the shield. Alternatively both cooperating surfaces may be inclined.

In alternative exemplary embodiments the rotational movement is generated by a cam-follower mechanism. It is to be noted that in the context of the present invention the term “cam-follower mechanism” covers both the “traditional” embodiment in which a cam (or slot or track) member is actively moved with the follower being the passively moved member, as well as the “re-versed” embodiment in which the “follower” is actively moved with the cam member being the passively moved member.

In an exemplary embodiment when the shield is moved from the retracted to the extended position, the needle hub rotates relative to the shield to a rotational locking position in which the shield cannot be retracted, this preventing reuse of the needle unit.

The coupling may be in the form of a mounting coupling allowing the needle unit to be mounted on a drug delivery device. The mounting coupling may comprise deflectable gripping fingers forming part of the needle hub and be adapted to grip a corresponding coupling structure on the drug delivery device, as well as blocking means forming part of the shield and being adapted to block radial movement of the gripping fingers corresponding to a lock state.

In an exemplary embodiment the needle unit is provided in combination with a drug delivery device to form an assembly, the drug delivery device comprising a housing comprising or being adapted to comprise a drug-filled cartridge, an actuatable expelling mechanism for expelling a pre-set or user-set amount of drug from the cartridge, and coupling means allowing the needle unit to be mounted on the drug delivery device, the shield being mounted non-rotatable relative to the drug delivery device. The latter may be provided with a cartridge mount, e.g. as part of a cartridge holder, allowing the needle unit to be mounted in fluid communication with the interior of the cartridge.

The mounting coupling may be actuatable between a lock state in which a mounted needle unit cannot be removed from the drug delivery device, and an actuated release state in which the needle unit can be removed from the drug delivery device, wherein the mounting coupling is actuated from the lock state to the release state by rotational movement of the needle hub relative to the shield.

The mounting coupling may comprise deflectable gripping fingers forming part of the needle hub and being adapted to grip a corresponding coupling structure on the drug delivery device, and blocking means forming part of the shield and adapted to block radial movement of the gripping fingers, corresponding to the lock state, wherein the deflectable gripping fingers are rotated from a lock position to a release position in which radial movement of the gripping fingers is possible, this allowing the needle unit to be removed from the drug delivery device.

In such an assembly, when the needle unit is mounted on the drug delivery device, the shield may be adapted to actuate the expelling mechanism actuation means when the shield is moved from the extended position to the retracted position, and when the shield is moved from the retracted to the extended position the needle unit is actuated to the locked state in which the shield cannot be retracted. By this arrangement double-actuation of the expelling mechanism with the same needle assembly mounted can be prevented.

The drug delivery device may comprise an actuator associated with the cartridge mount, wherein the actuator is adapted to actuate the mounting coupling from the initial mounting state to the lock state. The actuator may engage the shield of a mounted needle assembly to provide a distally directed axial force.

In a further exemplary embodiment a drug delivery assembly comprising a needle unit with a coupling as described above is provided. The drug delivery assembly further comprises a housing comprising or adapted to comprise a drug-filled cartridge, an actuatable expelling mechanism for expelling a pre-set or user-set amount of drug from the cartridge. The coupling means allows the needle hub to rotate relative to the housing and the shield to move axially and non-rotatable relative to the housing. The needle unit may be adapted to be non-removable from the housing. If the assembly is provided as a prefilled and preassembled disposable system, the needle unit is adapted to be actuated to move the needle into fluid communication with the drug-filled cartridge.

In a further aspect of the invention a drug delivery assembly is provided, comprising a housing comprising or adapted to comprise a drug-filled cartridge, and an actuatable expelling mechanism for expelling a pre-set or user-set amount of drug from the cartridge. The drug delivery assembly further comprises a needle hub, a hollow needle mounted non-rotatable in the needle hub and having a pointed distal end protruding from the needle hub, the hollow needle defining a reference axis and being adapted to allow drug to be expelled from the cartridge, and a shield in which the needle hub is fully or partly arranged, the shield being axially moveable relative to the needle hub between an extended position in which the shield axially covers the needle distal end, and a retracted position in which the needle distal end protrudes from the shield corresponding to a needle free length. The shield is mounted non-rotatable relative to the housing, with the needle hub being rotatable relative to the shield and thus to the housing. The needle hub is rotated relative to the shield when the shield is moved from its retracted to its extended position, and rotational movement of the needle hub takes place after at least 25%, at least 50% or 100% of the needle free length has been covered by the shield.

In an exemplary embodiment the drug delivery assembly comprises a drug delivery device and a thereon mountable needle unit. The needle unit comprises the needle hub, the hollow needle, the shield, and a mounting coupling allowing the needle unit to be mounted on the drug delivery device, the shield being mounted non-rotatable on the drug delivery device. The drug delivery device comprises the housing, and the actuatable expelling mechanism.

When the shield is moved from the retracted to the extended position, the needle hub may rotate relative to the shield to a rotational locking position in which the shield cannot be retracted.

Alternatively or additionally, the mounting coupling may be actuatable between a lock state in which a mounted needle unit cannot be removed from the drug delivery device, and an actuated release state in which the needle unit can be removed from the drug delivery device, wherein the mounting coupling is actuated from the lock state to the release state by rotational movement of the needle hub relative to the shield.

In an exemplary embodiment the mounting coupling comprises deflectable, e.g. flexible, gripping fingers forming part of the needle hub and adapted to grip a corresponding coupling structure, e.g. a fully or partly circumferential flange, on the drug delivery device, as well as blocking means forming part of the shield and adapted to block radial movement of the gripping fingers, corresponding to the lock state. The deflectable gripping fingers are rotated from a lock position to a release position in which radial movement of the gripping fingers is possible, this allowing the needle unit to be removed from the drug delivery device.

In an exemplary embodiment the mounting coupling is further being actuatable between an initial mounting state, e.g. as supplied to a user, in which the needle unit can be mounted on the drug delivery device, and the lock state. To achieve this functionality the mounting coupling may be actuated from the mounting state to the lock state by axial movement of the shield relative to the needle hub from an initial delivery position to an actuated position. The latter may correspond to the extended position. The shield may be moved axially by biasing means, e.g. a spring member, comprised in the drug delivery device, such a biasing member also serving to return the shield to its extended position after use.

As disclosed above, the needle unit comprises a mounting coupling allowing the needle unit to be mounted on the drug delivery device. Correspondingly, the latter may be provided with a cartridge mount, e.g. as part of a cartridge holder, allowing the needle unit to be mounted in fluid communication with the interior of the cartridge.

The drug delivery device may comprise a cartridge holder comprising or being adapted to receive a drug-filled cartridge, an actuatable expelling mechanism for expelling a pre-set or user-set amount of drug from the cartridge, a drive spring for driving the expelling mechanism, and actuation means for actuating the expelling mechanism, e.g. a proximally arranged release button.

In such an assembly, when the needle unit is mounted on the drug delivery device, the shield may be adapted to actuate the expelling mechanism actuation means when the shield is moved from the extended position to the retracted position, and when the shield is moved from the retracted to the extended position the needle unit is actuated to the locked state in which the shield cannot be retracted. By this arrangement double-actuation of the expelling mechanism with the same needle assembly mounted can be prevented.

The drug delivery device may comprise an actuator associated with the cartridge mount, wherein the actuator is adapted to actuate the mounting coupling from the initial mounting state to the lock state. The actuator may engage the shield of a mounted needle assembly to provide a distally directed axial force.

In a further aspect of the invention a needle unit is provided, comprising a needle hub, a hollow needle mounted in the needle hub and having a pointed distal end protruding from the needle hub, the hollow needle defining a reference axis, a shield in which the needle hub is fully or partly arranged, the shield being axially moveable relative to the needle hub between an extended position in which the shield axially covers the needle distal end, and a retracted position in which the needle distal end protrudes from the shield corresponding to a needle free length, and a mounting coupling allowing the needle unit to be mounted on a drug delivery device. The needle hub is rotated relative to the shield when the shield is moved from its retracted to its extended position, such that rotational movement of the needle hub relative to the shield takes place after at least 25%, at least 50% or 100% of the needle free length has been covered by the shield.

When the shield is moved from the retracted to the extended position, the needle hub may rotate relative to the shield to a rotational locking position in which the shield cannot be retracted.

In an exemplary embodiment the needle unit comprises a mounting coupling allowing the needle unit to be mounted on a drug delivery device. The mounting coupling comprises deflectable gripping fingers forming part of the needle hub and adapted to grip a corresponding coupling structure on the drug delivery device, as well as blocking means forming part of the shield and adapted to block radial movement of the gripping fingers, corresponding to a lock state. The deflectable gripping fingers and the blocking means are rotatable relative to each other from a lock position, corresponding to the lock state, to a release position in which radial movement of the gripping fingers is possible, this allowing the needle unit to be removed from the drug delivery device.

In further exemplary embodiments the needle unit is provided with a container adapted to accommodate the shield and the therein arranged hub, the needle assembly comprising a snap coupling between the container and the shield. The snap coupling is actuatable between a first state in which the needle unit can be removed from the container using a first amount of force, and a second state in which the needle unit can be removed from the container using a second higher amount of force. Indeed, the purpose of the second state is not to make it more difficult to remove the container but to prevent it and thus allow the container to be used as a pulling aid for removal of the hub from the cartridge mount.

The container may comprise an open end adapted to be sealed with a flexible foil member thereby providing a sealed, sterile interior for the needle assembly when supplied to the user.

The snap coupling may be provided with cooperating first and second coupling means arranged on the shield respectively the container, wherein the first coupling means is actuatable between an initial state corresponding to the snap coupling first state, and an actuated state corresponding to the snap coupling second state, and the first coupling means is actuated from the initial to the actuated state when the shield is moved from its extended to its retracted and back to its extended position.

In an exemplary embodiment the first coupling means comprises blocking means moveable relative to the flexible shield snap structures from a non-blocking position allowing radial movement of the flexible shield snap structures corresponding to the first state, to a blocking position in which radial movement of the flexible shield snap structures corresponding to the first state is prevented.

The blocking means may form part of the needle hub such that when the needle hub and the shield are rotated relative to each other when the shield is moved from its retracted to its extended position, then the first coupling means is actuated from the initial to the actuated state when the needle hub is rotated relative to the shield from an initial to an actuated position.

In all of the above-described embodiments the hollow needle may comprise a pointed proximal end adapted to be inserted through a needle-penetratable cartridge septum to provide fluid communication between the cartridge interior and the needle distal end outlet opening when the needle unit is mounted on a drug delivery device. To reduce the risk of accidental needle stick the proximal needle end may be arranged distally of the proximal-most portions of the needle hub and/or the shield, i.e. the distal needle end is arranged inside the needle unit.

As used herein, the term “drug” is meant to encompass any drug-containing flowable medicine capable of being passed through a delivery means such as a hypodermic needle in a controlled manner, such as a liquid, solution, gel or fine suspension. The drug may have a blood glucose controlling effect, e.g. human insulin and analogues thereof as well as non-insulins such as GLP-1 and analogues thereof.

### BRIEF DESCRIPTION OF THE DRAWINGS

In the following embodiments of the invention will be described with reference to the drawings, wherein:

FIG. 1 shows the components of an exemplary first embodiment of a needle assembly comprising a shield member, a hub member and a container,

FIG. 2 shows in a cross-sectional view the needle assembly of FIG. 1 in an assembled state with a needle unit formed by the shield member and the hub member mounted in the container,

FIG. 3 shows an alternative embodiment of a needle unit mounted on a pen device,

FIGS. 4A and 4B show the shield member of FIG. 1 seen from the proximal respectively the distal end,

FIG. 4C shows in a cross-sectional view the shield member of FIG. 4A,

FIGS. 5A and 5B show the hub member of FIG. 1 seen from the proximal respectively the distal end,

FIGS. 6A and 6B show the container of fig, 1 seen from the proximal respectively the distal end,

FIG. 6C shows in a cross-sectional view the shield member of FIG. 6B,

FIGS. 7-10 show in cross-sectional views different states of the needle assembly of FIG. 1 being mounted on a cartridge mount,

FIGS. 11, 12 and 14 show in cross-sectional views different states of the needle unit of FIG. 1 during operation,

FIG. 13 shows with the shield member outer portion cut away the needle unit of FIG. 14,

FIG. 15 shows in a cross-sectional detail view the shield lock,

FIGS. 16-18 show in cross-sectional views different states of the needle assembly of FIG. 1 being removed from the cartridge mount,

FIG. 19 shows an alternative embodiment of a cartridge mount to be used in combination with the needle unit of FIG. 1,

FIG. 20 shows a second embodiment of a drug delivery device with a mounted needle unit,

FIG. 21 shows in a cross-sectional view the drug delivery device 1 with the needle unit being replaced with a cap,

FIG. 22 shows an exploded view of the components of the drug delivery assembly of FIGS. 20 and 21,

FIGS. 23A and 23B show a perspective respectively a cross-sectional view of the shield member of FIG. 22,

FIGS. 24A and 24B show a perspective respectively a cross-sectional view of the hub member of FIG. 22,

FIGS. 25A and 25B show a perspective respectively a cross-sectional view of a container for the needle unit of FIG. 20,

FIG. 26 shows a cross-sectional view of the needle unit of FIG. 20 arranged in the container of FIG. 25A,

FIGS. 27A-27J show in a series of cross-sectional views mounting, actuation and removal of the needle unit on the drug delivery device,

FIG. 27CX and 27GX show cut-away views of the corresponding FIGS. 27C and 27G, and

FIGS. 28A-28C show in a series of cut-away perspective views movement of the shield and needle hub relative to a housing indicator opening during actuation of the needle unit.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The term “distal” refers to a portion of an element, assembly or device which during use is oriented towards a skin surface of a user, the term “proximal” referring to the opposed portion. Correspondingly, for a traditional pen drug delivery device the needle is arranged at the distal end and an end-mounted release button is arranged at the proximal end. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The term “assembly” does not imply that the described components necessarily can be assembled to provide a unitary or functional assembly during a given assembly procedure but is merely used to describe components grouped together as being functionally more closely related.

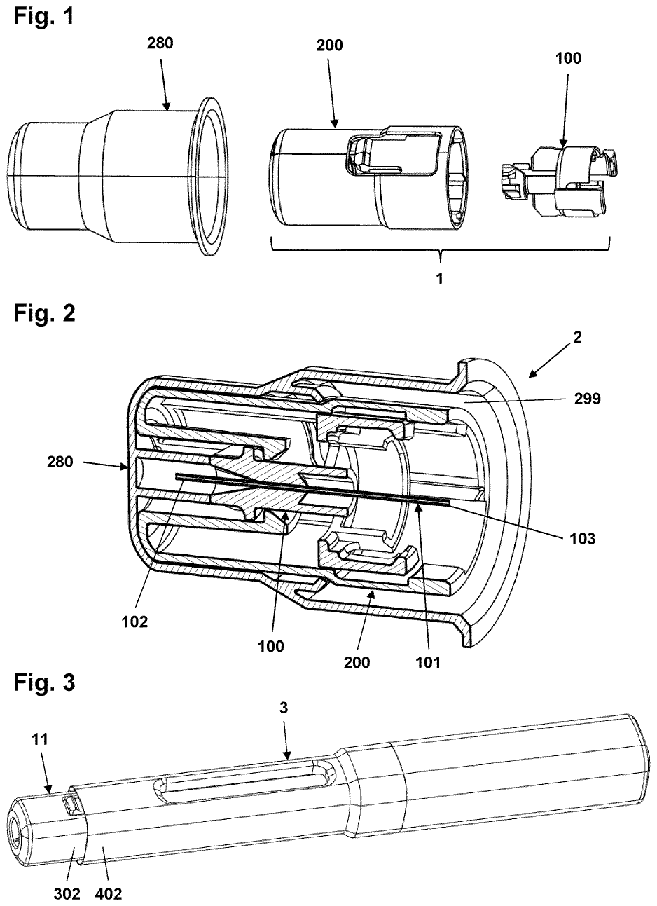

With reference to FIGS. 1 and 2 a first embodiment of a shielded needle assembly 2 is shown, comprising a needle hub 100 adapted to be mounted on a corresponding needle mount on a drug delivery device, a subcutaneous hollow needle 101 mounted in the hub and comprising a pointed free distal end portion adapted to be inserted subcutaneously through the skin of a user and a pointed free proximal end portion adapted to be penetrate a pierceable drug cartridge septum, a shield member 200 in which the needle hub is arranged, as well as a container 280. The needle hub with the mounted needle and the shield member in combination form a needle unit 1. The subcutaneous hollow needle 101 comprises bevelled proximal and distal ends and is arranged in a hub bore and secured in place by e.g. adhesive. The container 280 is adapted to receive the needle unit to thereby form a needle assembly, the container having an open end adapted to be sealed with a flexible foil member thereby providing a sealed, sterile interior for the needle unit 2 when supplied to the user. In the shown embodiment the needle proximal end extends proximally of the hub proximal-most portion but does not extend proximally of the shield proximal-most portion, this reducing the risk of accidental needle contact.

FIG. 3 shows a needle unit 11 mounted on an injection device 3 adapted to releasably receive the needle unit. In contrast to the circular configuration of the needle unit of FIGS. 1 and 2, FIG. 3 shows an exemplary design in which the shield 302 has an outer squarish configuration allowing it to be received in the correspondingly formed distal cartridge portion 402 of the injection device 3. Alternatively other non-circular designs could be implemented, e.g. oval or triangular. As will be apparent from the below detailed description of an exemplary embodiment of a needle assembly the functionality of the assembly relies on rotational movement between the hub and the shield, this irrespective of the outer configuration of the shield.

As will be explained in greater detail below, the needle hub 100 (in the following also just “hub” or “hub member”) and the shield 200 member (in the following also just “shield”) comprise a number of interacting structures allowing the shield and hub to move axially and rotatably relative to each other in a controlled manner during use and operation of the needle unit. In the below-described embodiment the shield is rotationally locked relative to the cartridge mount and the hub is axially locked relative to the cartridge mount when the needle unit is mounted on the cartridge mount. Rotational movement of the hub is controlled by axial movement of the shield relative to the cartridge mount and thus the hub.

As will also be explained in greater detail below, the container and the shield member comprise a number of interacting structures allowing the container to be used as a mounting and removal tool for the needle unit during use in an efficient and user-friendly manner. The shield, hub and container generally comprise functional structures in opposed pairs, however, any suitable numbers of such structures could be used, e.g. one, two or three. The structures and functionality of the needle unit and the container are described with reference to an embodiment (see FIGS. 1, 2 and 4A-18) having a generally circular configuration of the shield and container, how-ever, the shield and container may have a non-circular configuration as shown in FIG. 3 in which the shield 302 and drug delivery device housing 402 have a squarish configuration.

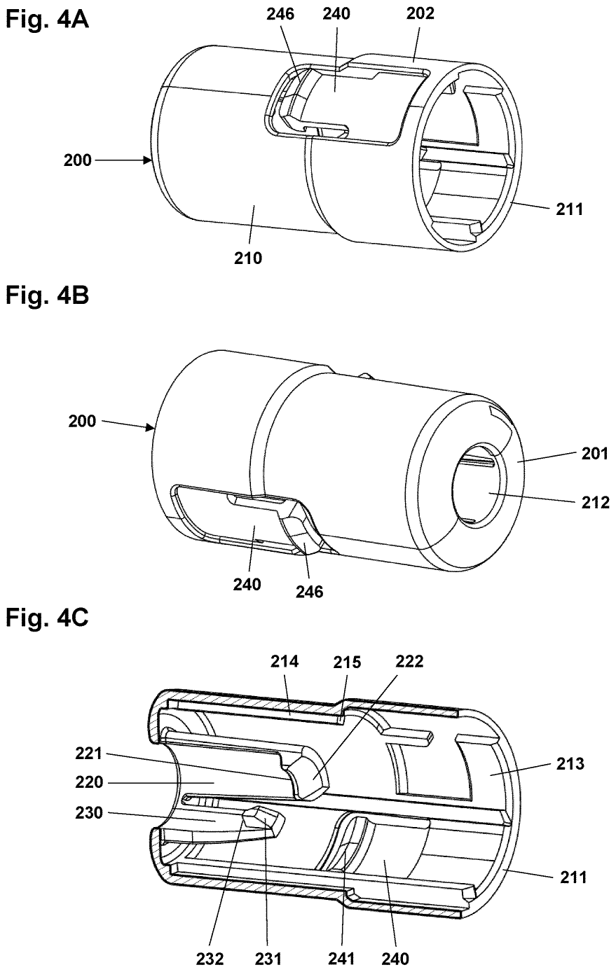

As shown in FIGS. 4A-4C the shield 200 has a generally tubular configuration with a circumferential outer wall 210, a proximal skirt portion 202 having a proximal opening with a circumferential edge 211, and a distal end surface 201 with a smaller distal opening 212 from which a tower structure protrudes axially inwardly. The tower structure comprises first and second pairs of opposed arms with an offset of 90 degrees in the shown embodiment. The first pair of arms is in the form of flexible stop arms 220 each having a distally facing axial stop surface 221 at the proximal free end adapted to engage corresponding proximally facing stop surfaces on the hub tower portion (see below) as well as a proximally facing ramp surface 222 used during assembling of the needle assembly. The second pair of arms is in the form of flexible control arms 230 each having a proximally facing ramp surface 231 and a distally facing control surface 232 at the proximal free end, the surfaces being adapted to engage corresponding distally facing ramp surfaces respectively proximally facing control surfaces on the hub tower portion (see below). At the proximal end the shield comprises a pair of opposed inner blocking surfaces 213 adapted to engage corresponding flexible hub arms (see below). The shield further comprises a pair of opposed locking ribs 214 on the shield inner wall surface, each rib having a proximally facing locking surface 215 adapted to engage a corresponding locking surface on the hub. The shield outer wall 210 is further provided with a pair of opposed flexible coupling arms 240, each arm comprising an inner ridge 241 and an outer ridge 246 at the distal free end adapted to engage corresponding structures on the hub respectively the container (see below).

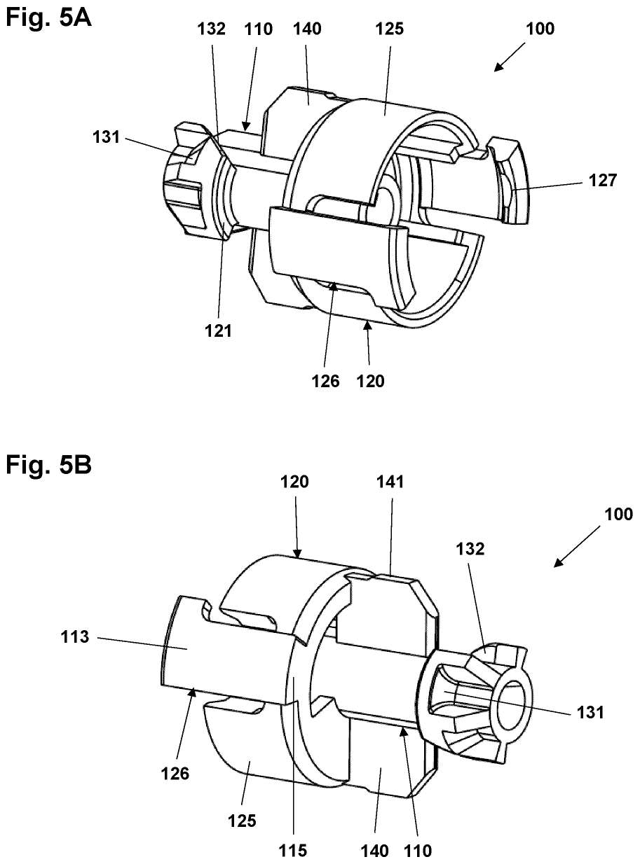

As shown in FIGS. 5A and 5B the hub 100 comprises a distal tower portion 110 comprising a central bore adapted to receive a subcutaneous needle as well as a proximal skirt portion 120. At the distal end the tower portion comprises three pairs of opposed function surfaces adapted to cooperate with corresponding surfaces on the shield. A pair of proximally facing stop surfaces 121 adapted to engage the distally facing stop surfaces 221 on the shield, a pair of distally facing ramp surfaces 131 adapted to engage the proximally facing ramp surfaces 231 on the shield, and a pair of inclined proximally facing control surfaces 132 adapted to engage the distally facing control surface 232 on the shield during operation. The hub tower further carries a pair of opposed axially extending blocking flanges 140 each having an outer blocking edge 141 adapted to engage the corresponding shield coupling arm inner ridge 241 during operation. The skirt portion 120 comprises a disc portion with two opposed part-circumferential bearing surfaces 125 extending proximally and being adapted to engage corresponding bearing structures on the shield to assure stability during axial and rotational movement between the hub and shield during operation. The skirt portion further comprises an opposed pair of proximally extending flexible coupling arms 126 each having an outer surface 113 adapted to engage the shield inner blocking surface 213 during operation, an inwardly facing snap coupling ridge 127 arranged at the free proximal end of the coupling arm and adapted to engage a corresponding coupling structure on a cartridge mount, as well as a distally facing lock surface 115 adapted to engage the proximally facing shield locking surface 215 during operation

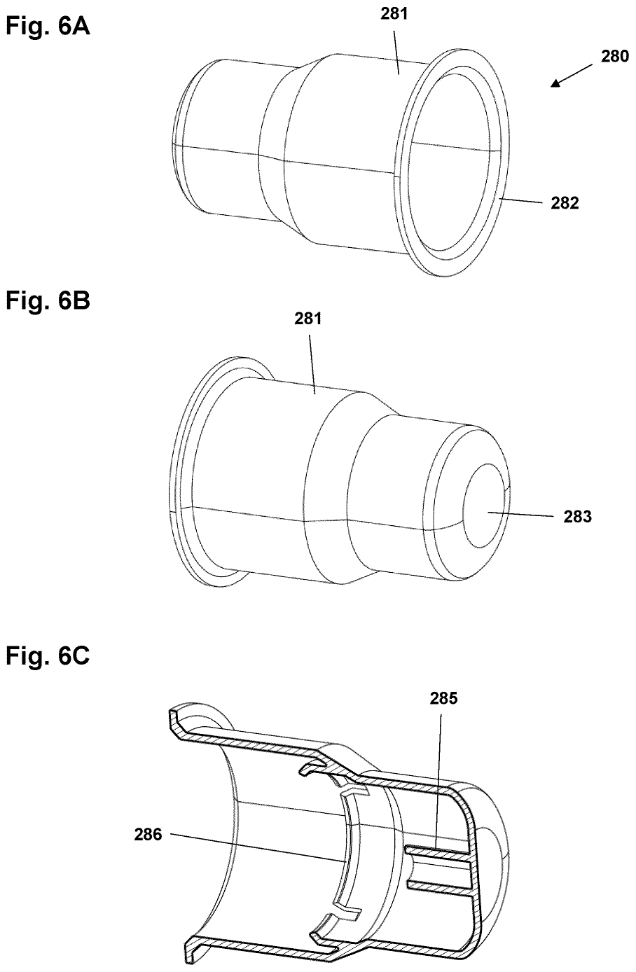

As shown in FIGS. 6A-6C the container 280 has a generally tubular configuration with a circumferential outer wall 281, a proximal opening with a circumferential flange 282 and a closed distal end 283 from which a tower structure 285 protrudes axially inwardly. The container has a stepped configuration with a smaller-diameter distal portion and a larger-diameter proximal portion. An inner circumferential ridge structure 286 is arranged between the two portions and adapted to engage the outer ridges 246 on the shield flexible coupling arms 240. The remaining part of the circumferential ridge serves to support the shield when arranged in the container. With the shield mounted in the container the larger-diameter proximal portion provides a circumferential space 299 (see FIG. 2) between the container and the shield allowing a correspondingly shaped drug delivery housing portion to be received therein during mounting of the needle assembly on the drug delivery device.

During assembly a hollow subcutaneous needle with bevelled proximal and distal ends is arranged in the hub bore and secured in place, e.g. by means of adhesive, this providing a free distal end 102 and a free proximal end 103. The hub is subsequently inserted in the shield with the stop surfaces 121 and the ramp surfaces 131 rotationally aligned with the shield stop surfaces 221 respectively the shield ramp surfaces 231, this allowing the shield stop surfaces to snap into engagement with the hub stop surfaces 121. Also the hub coupling arms 126 are rotationally aligned with the shield blocking surfaces 213. The assembled needle unit is then inserted into the container with the container rim 286 snapping into engagement with the outer ridges 246 on the shield flexible coupling arms 240. As seen in FIG. 2 the container tower 285 abuts the hub tower distal end. As a final assemble step a flexible foil member is attached to the container proximal flange 282 thereby sealing the interior for subsequent sterilization.

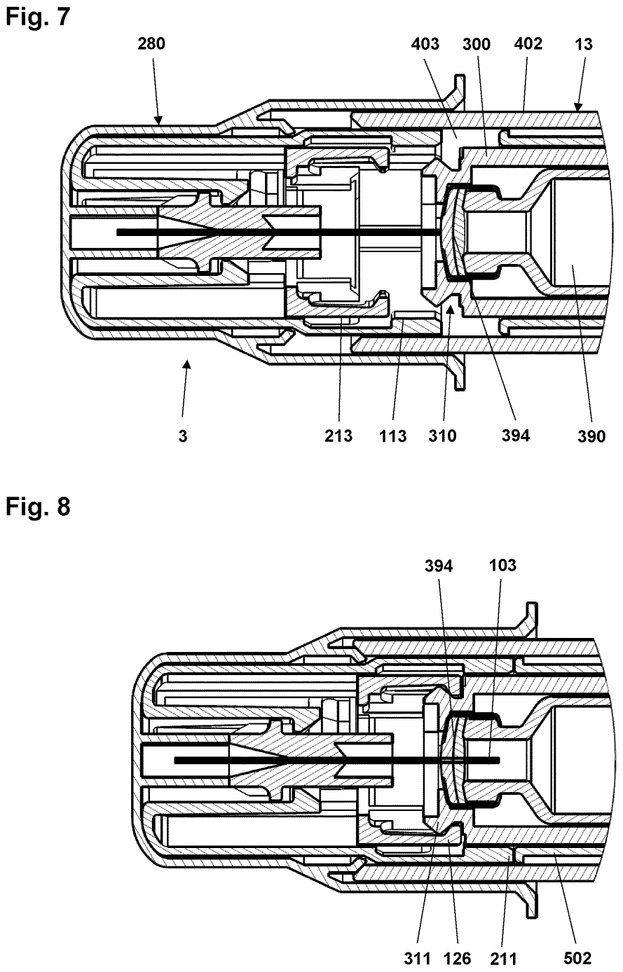

In the following the different features and aspects of the above-described needle unit and container combination will be described with reference to FIGS. 7-18 showing a needle unit being mounted on a corresponding drug delivery device, operated to allow an amount of fluid drug to be injected subcutaneously and subsequently removed from the drug delivery device.

After the flexible seal foil has been removed from the container 280 by the user, the container can be used as a tool to mount the needle unit on a drug delivery device 13 with a corresponding cartridge mount 310, see FIG. 7. A drug-filled cartridge 390 with a septum 394 is arranged in a cartridge holder 300. In the shown embodiment the cartridge mount is arranged proximally of the distal end of the drug delivery device housing 402 with a circumferential space 403 between the cartridge holder 300 and the housing adapted to receive the shield proximal portion in non-rotational engagement, e.g. by cooperating splines or a non-circular form such as oval or squared off. Such a non-circular design would also make it easy for the user to orient the needle assembly rotationally correct relative to the drug delivery device. As shown in FIG. 7 the hub coupling is in an initial mounting state in which the shield blocking surfaces 213 are not engaging the flexible hub arm outer surfaces 113.

The container assures that the shield can be pushed firmly into engagement with the cartridge mount by the user, this allowing the free proximal needle end 103 to penetrate the cartridge septum 394 and the flexible hub coupling arms 126 to be initially moved radially outwards in a receiving space on the shield and subsequently snap radially inwards into engagement with corresponding snap coupling means 311 on the cartridge mount, see FIG. 8. In the shown embodiment the drug delivery device is provided with a pair of opposed spring-biased connector members 502 which initially are pushed proximally a short distance by the shield circumferential edge 211. During mounting a spline connection between the hub and shield prevents rotational movement therebetween.

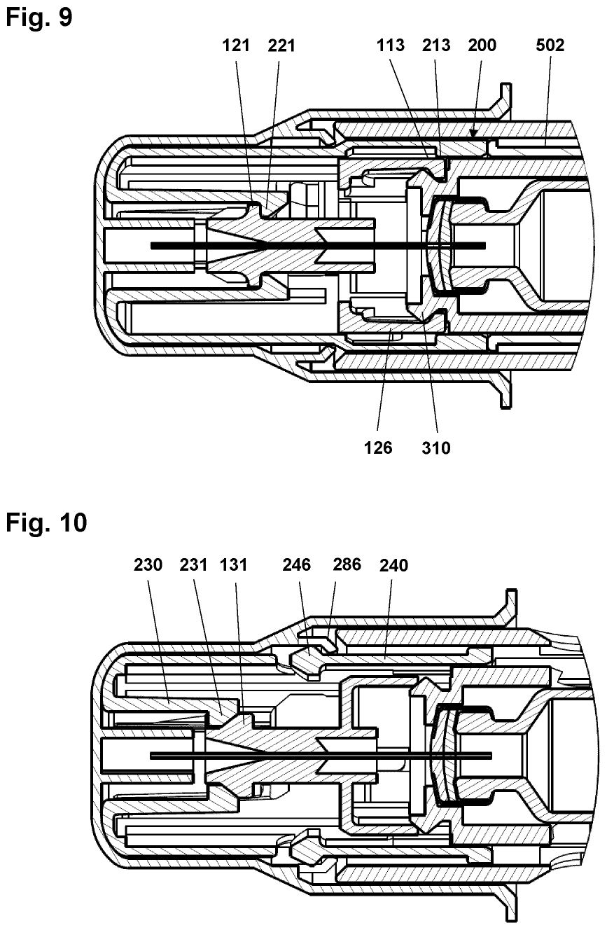

When the user stops pushing on the container (or starts to pull the container away) the spring-biased connector members 502 will push the shield 200 slightly distally until the distally facing axial stop surfaces 221 on the shield tower engage the corresponding proximally facing stop surfaces 121 on the hub tower portion. As the shield is moved distally the shield blocking surfaces 213 will move into engagement with the flexible hub arm outer surfaces 113 and thereby prevent radial outwards movement thereof, this securely locking the hub 100 to the cartridge mount 310 corresponding to an actuated hub coupling lock state in which a mounted needle unit cannot be removed from the drug delivery device, see FIG. 9.

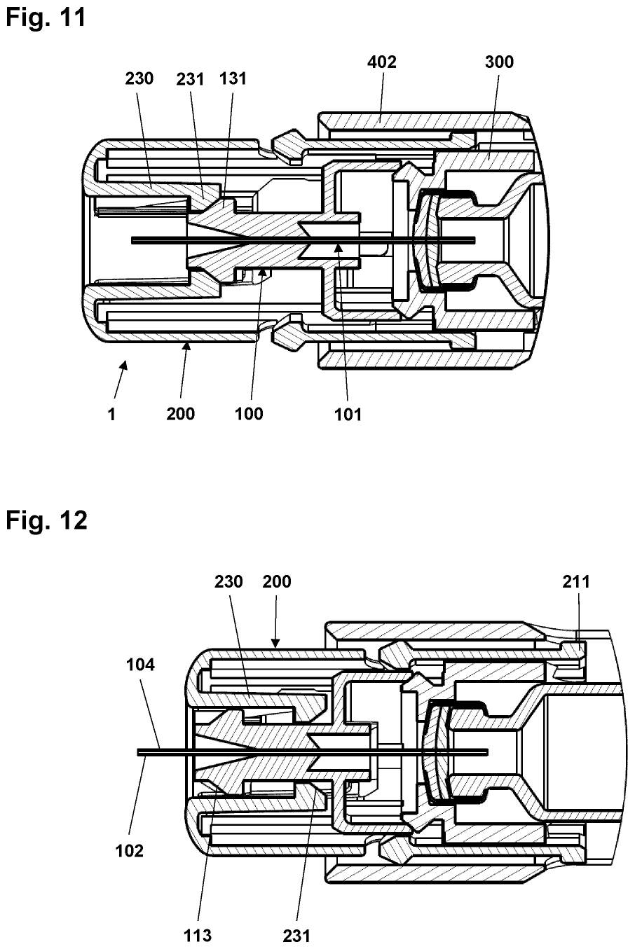

As the user pulls the container 280 further distally to fully remove it, the container snap coupling ridge will disengage the shield 200. In the shown embodiment the shield comprises a pair of flexible coupling arms 240 each with an outwardly facing ridge 246 at the free end which can be forced inwardly by the container snap coupling ridge 286 thereby allowing the container to be moved distally with ease, see FIG. 10. When the container is fully removed the drug delivery device with the mounted needle unit 1 is ready for use as shown in FIG. 11. It should be noted that the cross-sectional views of FIGS. 10-12 have been rotated 90 degrees when compared with FIGS. 7-9, whereby the ramp surfaces 231 on the shield flexible control arms 230 can be seen in resting engagement with the distally facing ramp surfaces 131 on the hub tower. Further, due to the rotation the connectors 502 engaging the shield proximal end 211 cannot be seen. In this state the shield is in its initial extended position.

When the user pushes the needle unit towards a skin surface the shield 200 is pushed proximally allowing the needle distal end 102 to be inserted subcutaneously. During initial proximal movement of the shield the ramp surfaces 231 on the flexible control arms 230 are pushed over the hub tower ramp surfaces 131. As the shield is moved further proximally to its fully retracted position, the needle distal end protrudes from the shield corresponding to a needle free length portion 104 (see FIG. 12). At the same time the shield proximal circumferential edge 211 pushes the pair of connector members 502 proximally (see FIG. 9) to thereby release the drug delivery device expelling mechanism thereby starting subcutaneous injection. As appears, the connector members 502 serve both as a locking actuator for the hub coupling and as release members for the expelling mechanism. A drug delivery device comprising a similar release connector adapted to transfer translational movement from a telescopically movable needle shield to an expelling mechanism is disclosed in WO 2021/122219 and WO 2021/122192 and would be suitable for adaptation to also function with the present needle unit. Alternatively, the expelling mechanism may be released by e.g. manually operated release means on the drug delivery device such as a proximally arranged push button. Such a push button may be blocked for actuation until released by proximal movement of the shield.

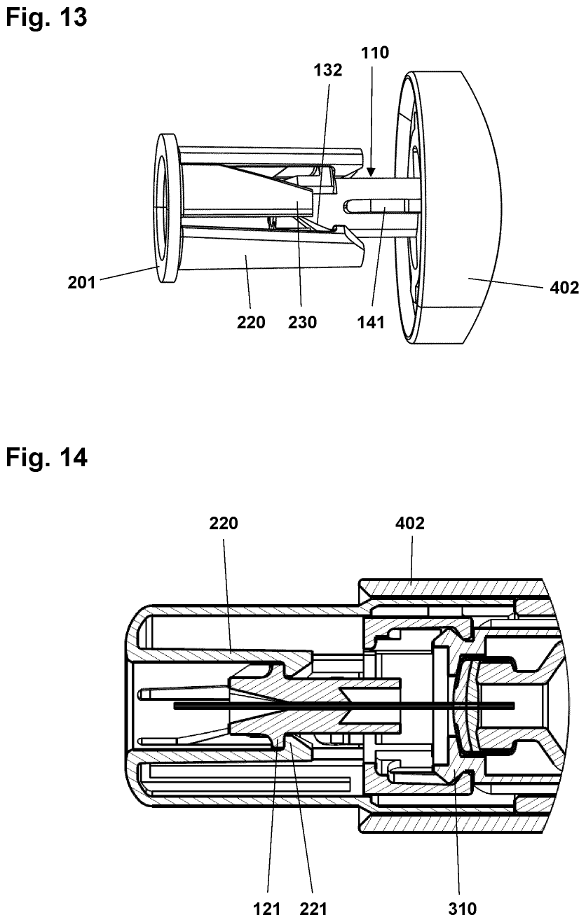

After the dose has been fully expelled the user withdraws the needle unit from the skin surface thereby allowing the spring-biased connectors 502 to move the shield 200 distally to its fully extended final position again covering the needle distal portion 102. During this movement the control surfaces 232 (see FIG. 4C) on the shield control arms 230 will engage with the inclined proximally facing control surfaces 132 on the hub tower, which will force the needle hub 100 to be rotated as the shield is rotationally locked to the drug delivery device, see FIG. 13. In the shown embodiment the hub is rotated 45 degrees relative to the shield. In the shown embodiment the initial and final extended positions of the shield relative to the hub are the same.

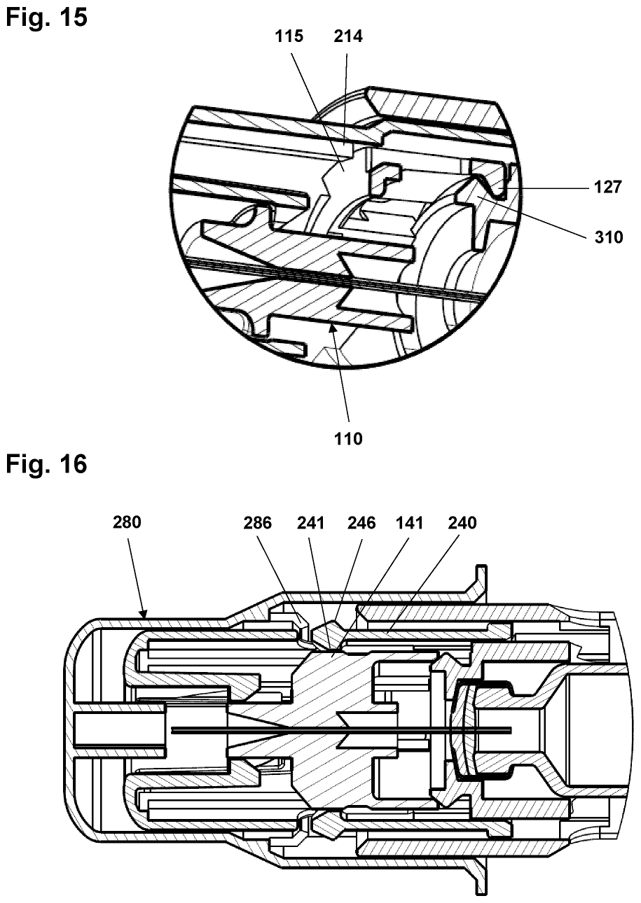

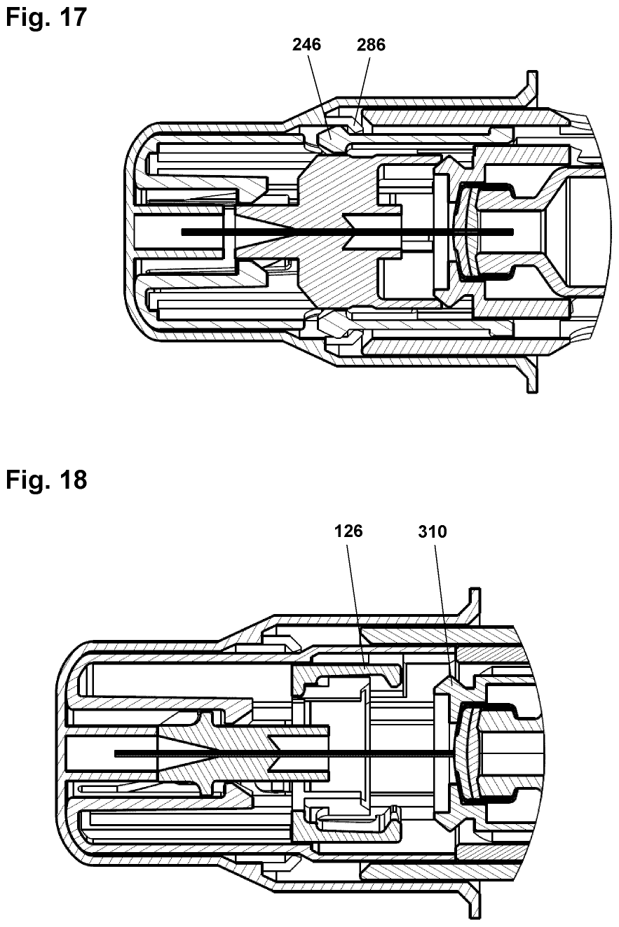

During rotation of the hub 100 relative to the shield 200 a number of structures are moved into and out of engagement with each other. In the following it should be noticed that the cross-sectional views of FIGS. 16 and 17 have been rotated 90 degrees when compared with FIGS. 14 and 18, this allowing the cooperation between the hub blocking flange edges 141 and the shield flexible coupling arm inner ridges 241 to be shown.
(i) As the hub rotates the flexible hub coupling arms 126 rotate out of engagement with the shield blocking surfaces 213 thereby allowing the flexible hub arms to move radially outwards, see FIG. 14, and thus the hub 100 to be removed from the cartridge mount 310.
(ii) As the needle 101 is fixed in the hub 100 it follows that the needle will rotate with the rotating hub, however, it may not be desirable that the needle rotates when fully inserted subcutaneously. Correspondingly, the hub and shield can be designed with an axial slack or “play” before the shield control surfaces 232 engages the inclined hub control surfaces 132, this allowing the needle to be at least partly withdrawn from the skin before rotation starts. Indeed, the later rotation starts the steeper the inclination of the hub control surface would have to be.
(iii) Before operation of the needle unit was initiated, the pair of locking ribs 214 on the shield inner surface was free to move in the axial direction thus allowing the shield to be moved from its extended to its retracted position. As the hub is rotated the pair of distally facing locking surfaces 115 are rotated into alignment with the proximal ends 215 of the locking ribs 214 thereby preventing repeated retraction of the shield and thus use of the needle unit, thereby providing a safety lock, see FIG. 15. Correspondingly, the locking surfaces should be designed to withstand relatively large forces to prevent re-activation of the needle unit, e.g. if the pen device is dropped on a solid surface or in a misuse scenario. Further, in case the drug delivery device is shield-released the shield lock will also serve as a double dose prevention means.
(iv) As the hub 100 rotates the pair of blocking flange edges 141 are rotated into alignment with the shield flexible coupling arm inner ridges 241 thereby preventing the arms from flexing inwardly. In this state the actuated and locked needle unit can be removed from the cartridge mount by simply gripping and pulling the shield distally (the shield being axially coupled to the hub via the corresponding stop surfaces 221, 121 on the shield tower respectively the hub tower, see FIG. 14), however, in the shown embodiment the container is intended to be used as a tool also for removing the needle unit, see FIG. 16. More specifically, with the shield flexible coupling arms 240 in a blocked state the container 280 is mounted on the shield by the user applying axial force until the container snap coupling ridge 286 overrides the shield outer ridges 246, e.g. by ovalizing the container wall, see FIG. 17. The container snap coupling is designed to have a release force larger than the release force necessary to pull the flexible hub coupling arms 126 axially out of engagement with the cartridge mount 310, this allowing the needle unit to be removed from the cartridge mount securely held in the container after which it can be safely discarded, see FIG. 18. As the needle unit is locked to the container via the container snap coupling and the proximal end is positioned a certain distance inside the container and only being surrounded by a small free space, removal of the needle unit from the container would be difficult for the user.

### Alternative Embodiments

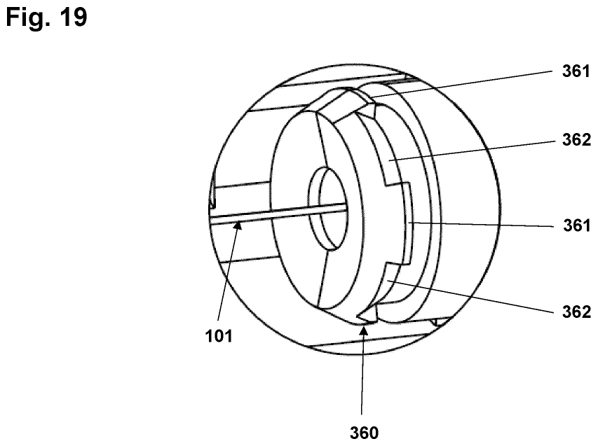

As the hub rotates relative to the cartridge mount this property can be utilized to provide a cartridge mount 360 having different snap coupling properties depending on the hub's rotational position relative to the cartridge mount. More specifically, as shown in FIG. 19 the cartridge mount may be provided with a first set of snap coupling structures 361 adapted to engage the hub snap coupling structures allowing the hub to be mounted using a first amount of force, and a second set of snap coupling structures 362 adapted to engage the hub snap coupling structures allowing the hub to be removed using a second lower amount of force, wherein the first and second cartridge mount snap coupling structures are rotationally offset relative to each other, e.g. by 45 degrees. A given coupling force may be provided by e.g. the inclination of coupling ramp surfaces.

As an alternative to the shield flexible coupling arms the shield outer wall may be provided with a thinned wall portion carrying a snap protuberance, the thin wall portions allowing the snap protuberances to flex inwardly when not blocked by the hub. Such a design would provide a cleaner visual appearance and prevent potential ingress of matter that may interfere with the shield mechanism.

As a further alternative the shield coupling may be provided with one or more radially moveable shield snap structures, and the container coupling means comprises corresponding container snap structures adapted to engage the shield snap structures. Such radially moveable shield snap structures may be located on a thin-walled shield portion, or on a flexible finger structure. The shield coupling means comprises actuating means moveable relative to the radially moveable shield snap structures from a non-engaging position corresponding to the first state, to an engaging position in which the shield snap structures are moved radially outwards corresponding to the second state. The actuating means may correspond to the above-described flanges 141. As appears, a stronger snap coupling can be provided by e.g. either making the shield coupling structure stiffer, or by making it to protrude further outwardly.

As a yet further alternative the blocking mechanism for the shield-container coupling may be dispensed with, i.e. the hub does not comprise structures engaging the shield-container coupling during operation. In this way the force required to remove, mount and prevent removal of the container from the shield during operation is provided only by design of the cooperating surfaces, e.g. the slope of ramp surfaces.

In case it is not desirable to have a rotating needle hub, an additional component can be added to achieve a safety lock in a similar way. More specifically, a needle unit may be provided with a hub and a shield coupled to each other to allow axial movement only, the assembly comprising a lock ring which during operation is rotated by the axial movements of the shield back and forth relative to the hub, the components comprising cooperating control surfaces adapted to rotate the lock ring from a non-blocking to a blocking position when the shield is returned from its retracted to its extended position.

In a further alternative arrangement, the shield final extended position is distal of the shield initial extended position, this allowing the shield coupling means to be actuated from the initial to the actuated state when the shield is moved from the retracted to the final extended position. A similar arrangement is utilized in the shielded needle assembly disclosed in above-mentioned WO 2018/215605 in which the shield is returned to a distal-most final position allowing a container to snap into engagement with the shield. When coupling actuation is based on hub rotation, the shield initial and final extended positions relative to the hub will typically be the same.

In a yet further alternative arrangement the needle assembly is provided with a snap coupling comprising cooperating first and second coupling means arranged on the shield respectively the container, this as described above, however, the container coupling means is actuatable between an initial state corresponding to the snap coupling first state, and an actuated state corresponding to the snap coupling second state. The container coupling means may be actuated from the initial to the actuated state when the container in a first rotational position relative to the shield is removed from the shield and subsequently reattached to the shield in a second rotational position. As appears, in such a simplified arrangement actuation of the snap coupling involves active manipulation of the shield by the user. To assure correct orientation of the container during use, the container and the shield (or drug delivery device) may be provided with corresponding indicia.

In a specific embodiment the second coupling means comprises first and second coupling structures, the first coupling structure being adapted to engage the first coupling means when the container is in the first rotational position relative to the shield, and the second coupling structure is adapted to engage the first coupling means when the container is in the second rotational position relative to the shield. The coupling structures may be in the form of more or less “aggressive” snap structures engaging the shield.

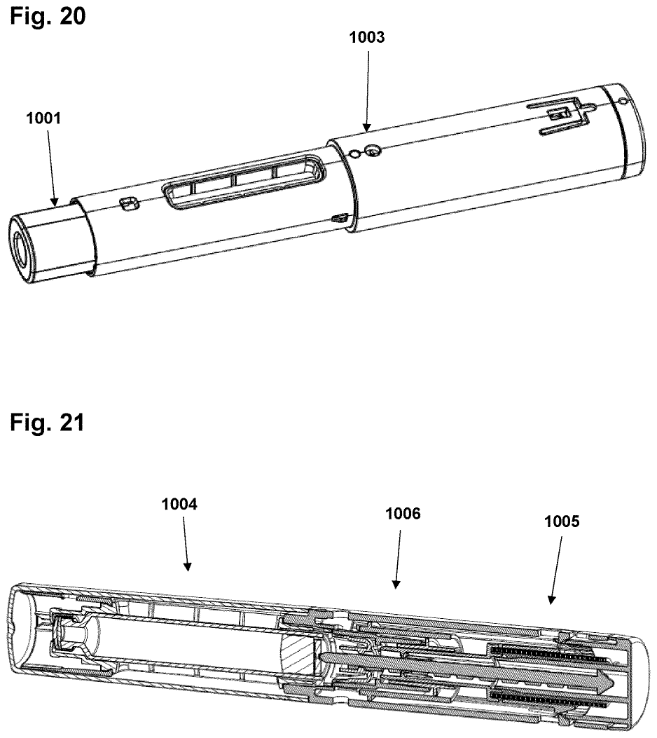

With reference to FIGS. 20-28C a further embodiment of a shielded needle assembly will be described.

With reference to FIG. 20 a drug delivery device 1003 with a mounted needle unit 1001 is shown. The device has a general tubular configuration defining a general reference axis. FIG. 21 shows in a cross-sectional view the drug delivery device 1003 with the needle unit being replaced with a cap. In the shown embodiment the cap fits snugly over the distal part of the drug delivery device and thus does not allow a needle unit to be mounted at the same time. The drug delivery device comprises a distal cartridge holder portion 1004 in which a drug filled cartridge is arranged, a proximal portion comprising a drive spring system 1005 as well as an intermediate portion comprising a control system 1006. A piston rod is arranged axially in the device and adapted to be moved distally by the drive spring to expel an amount of fluid drug through a mounted needle unit, the amount of axial travel of the piston rod being controlled by the control system. In FIG. 21 the actuator return spring is not shown.

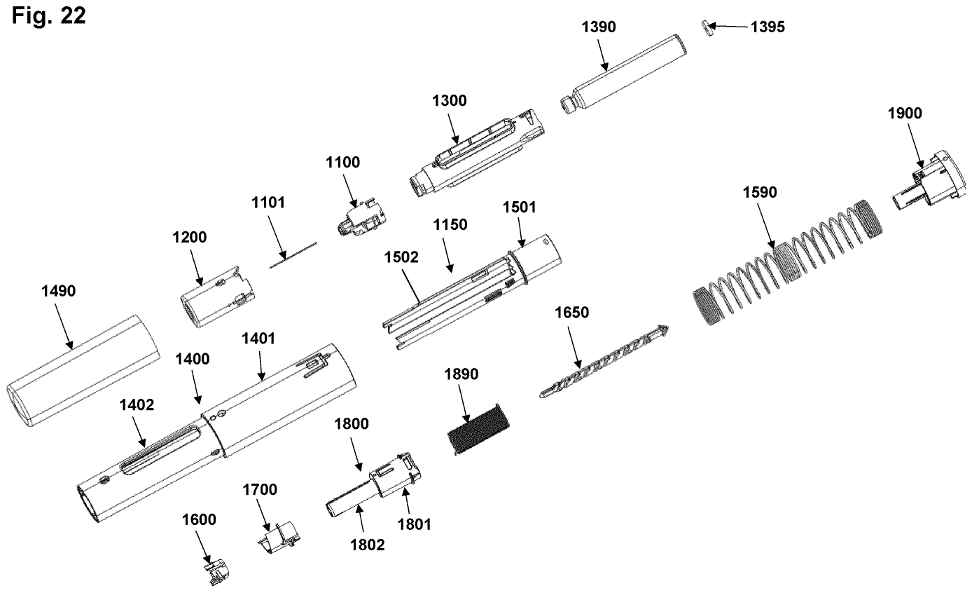

In the exploded view of FIG. 22 the individual components of the drug delivery device and needle unit are shown. The drug delivery device comprises a tubular housing 1400 with a proximal engine portion 1401 and a distal cartridge portion 1402 adapted to house a cartridge holder 1300 in which a drug cartridge 1390 is arranged, the drug cartridge comprising a distal outlet end with a needle pierceable septum, a proximal circumferential edge and an axially displace-able piston. A piston washer 1395 is arranged in the cartridge engaging the piston proximal surface. An actuator 1500 comprises a cylindrical proximal portion 1501 from which a pair of legs 1502 extend distally interposed between the housing and the cartridge holder. The drive nut 1600 is mounted in the housing and adapted to receive piston rod 1650 in threaded engagement. The piston rod is non-rotationally received in the distal tubular portion 1802 of drive member 1800 which is adapted to be rotationally driven by pre-strained drive spring 1890 arranged in the proximal portion 1801 of the drive member, the drive spring's proximal end being anchored to the housing via spring base 1900. Control member 1700 is in splined engagement with the drive member tubular portion 1802 and is adapted to be moved axially in and out of engagement with the housing to thereby control rotation of the drive member. A large-diameter return spring 1590 is arranged to provide a distally directed biasing force on the actuator. The drug delivery device is adapted to receive a needle unit at its distal end, the needle unit comprising a needle hub 1100 with a needle 1101, as well as a shield member 200 in which the needle hub is arranged in. When a needle unit is not mounted on the drug delivery device a cap 1490 can be mounted to cover the cartridge portion 1402.

A detailed description of the drug delivery device per se is disclosed in EP 23212595.5 which is hereby incorporated by reference. In the following only those portions of the drug delivery which directly engage with the needle unit will be described.

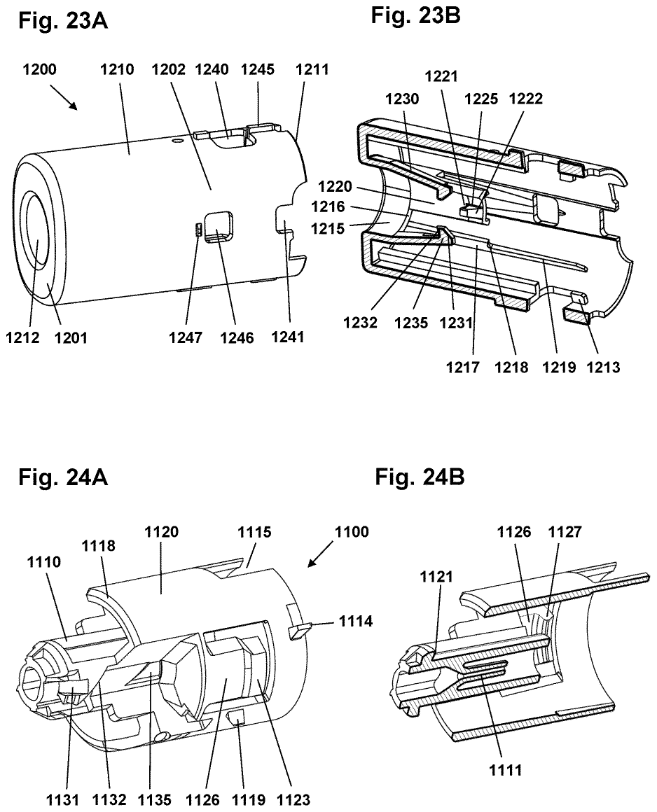

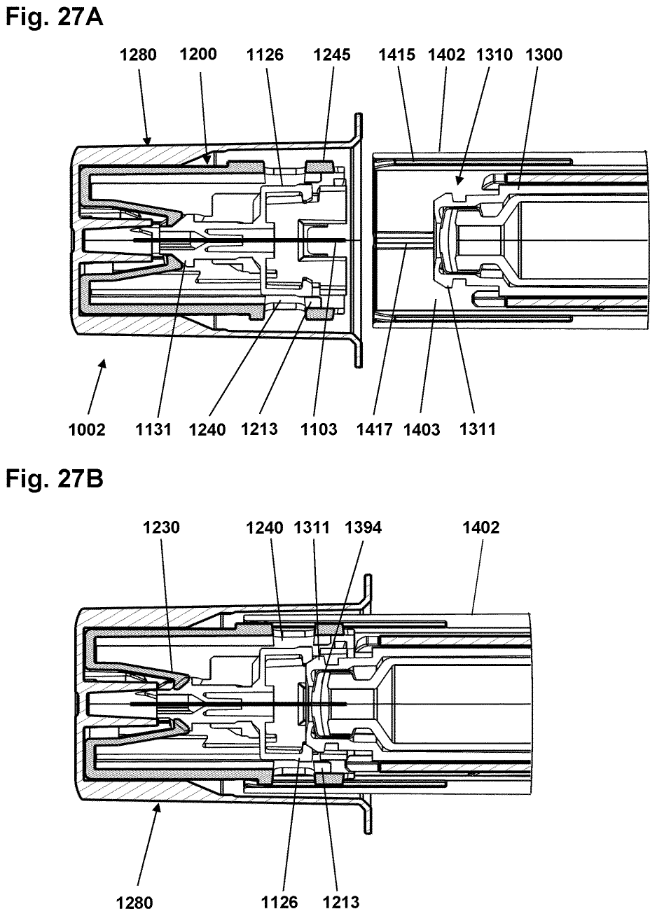

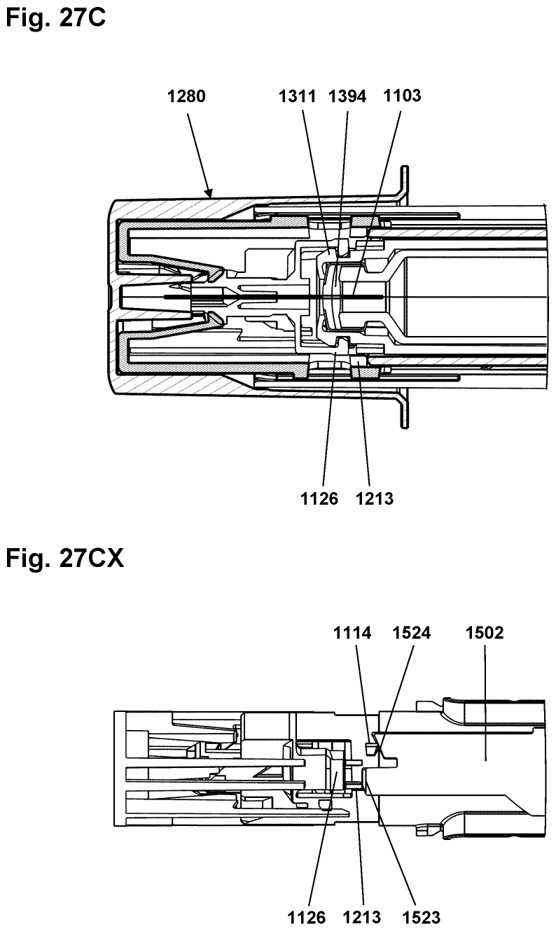

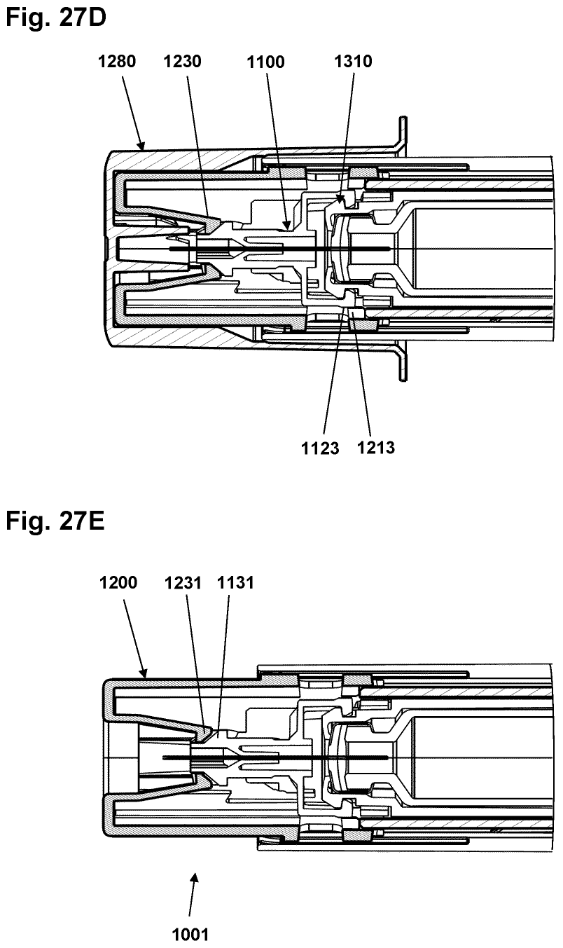

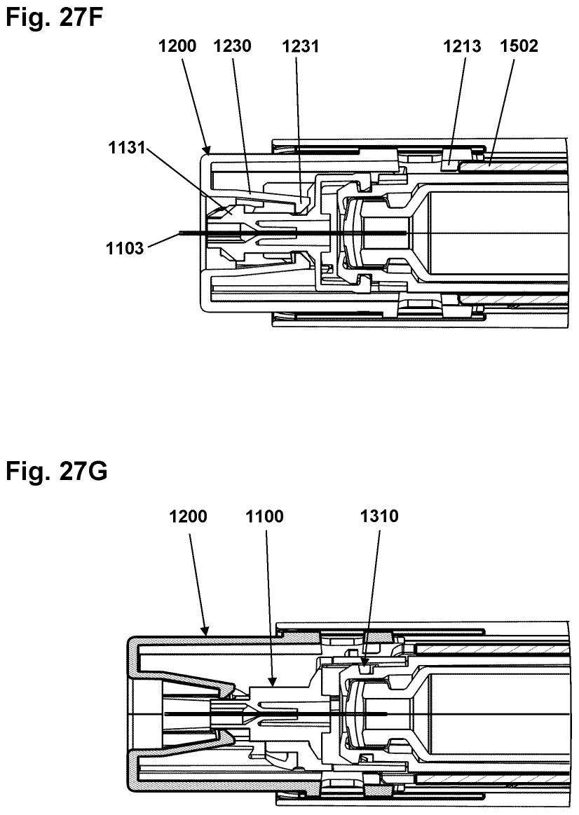

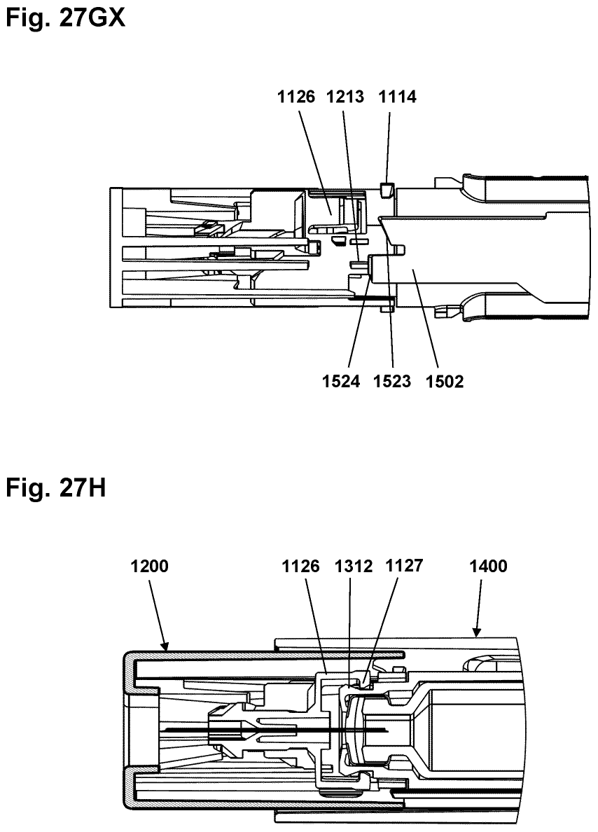

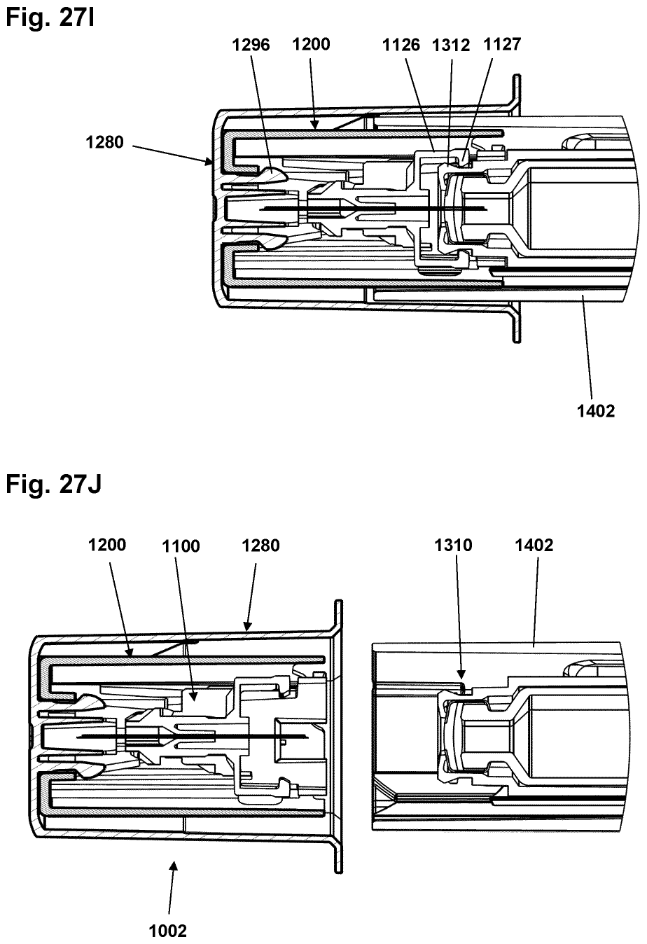

As shown in FIGS. 23A and 23B the shield 1200 has a generally tubular configuration with a circumferential outer wall 1210 having a proximal skirt portion 1202, a proximal opening with a circumferential edge 1211, and a distal end surface 1201 with a smaller distal opening 1212 from which a tower structure protrudes axially inwardly. The outer wall and thus also the proximal skirt portion have in the shown embodiment a circumferential non-circular configuration in the form of a super-elliptic cross-section. The tower structure comprises a circumferential skirt portion 1215 from which first and second pairs of opposed arms extend proximally with a rotational offset of 90 degrees in the shown embodiment. Between the arms the skirt portion comprises free grip edge portions 1216. The first pair of longer arms are in the form of flexible assembling arms 1220 each having a hook portion 1225 at the proximal free end with a distally facing axial stop surface 1221 adapted to engage corresponding proximally facing stop surfaces 1121 on the hub tower portion (see below) as well as a proximally facing ramp surface 1222 used during assembling of the needle unit. The second pair of shorter arms is in the form of flexible control arms 1230 each having a hook portion 1235 at the proximal free end with a proximally facing ramp surface 1231 and a distally facing control surface 1232, the surfaces being adapted to engage corresponding distally facing ramp surfaces respectively proximally facing control surfaces on the hub tower portion (see below). At the proximal end the shield comprises opposed pairs of inner actuation ribs 1213 adapted to engage corresponding flexible hub arms (see below). The actuation ribs 1213 also serve to lock the coupling arms (see below) and to centre the generally circular hub in the super-elliptic shield and thus assure stability during axial and rotational movement between the hub and shield during operation. The shield further comprises a pair of opposed locking ribs 1217 on the shield inner wall surface, each rib having a proximally facing locking surface 1218 adapted to engage a corresponding locking surface on the hub. The locking ribs proximally extend into lower torque ribs 1219 adapted to engage torque flanges on the hub (see below). The shield wall 1210 is further provided with an outer pair of opposed mounting ribs 1245 adapted to engage corresponding shield slots 1415 in the housing, a pair of opposed windows 1240 adapted to allow outwards movement of the hub coupling arms (see below), as well as a first indicator opening 1241 and a second indicator opening 1246. In the shown embodiment the first indicator opening 1241 is “open” as for design reasons the shield edge 1211 comprises cut-outs for structures in the drug delivery device. In the shown embodiment a further pair of opposed guiding structures 1247 adapted to engage corresponding shield guides 1417 in the housing (see FIG. 27A) is provided distally of the indicator openings 1246.

As shown in FIGS. 24A and 24B the hub 1100 comprises a distal tower portion 1110 comprising a central bore 1111 adapted to receive a subcutaneous needle as well as a proximal skirt portion 1120. At the distal end the tower portion comprises three pairs of opposed function surfaces adapted to cooperate with corresponding surfaces on the shield: (i) a pair of proximally facing stop surfaces 1121 adapted to engage the distally facing stop surfaces 1221 on the shield assembling arms, (ii) a pair of distally facing ramp surfaces 1131 adapted to engage the proximally facing ramp surfaces 1231 on the shield, and (iii) a pair of inclined proximally facing control surfaces 1132 adapted to engage the distally facing control surfaces 232 on the shield during operation. At the proximal end the tower portion comprises a pair of opposed snap indentations 1135 adapted to engage the control arm hook portions 1235. The skirt portion 1120 comprises an opposed pair of proximally extending flexible coupling arms 1126, each arm having an outer surface 123 adapted to engage the shield inner actuation ribs 1213 during operation, and an inwardly facing snap coupling ridge 1127 arranged at the free proximal end of the coupling arm and adapted to engage a corresponding coupling structure on the cartridge mount 1310. The skirt portion further comprises a pair of distally facing lock surfaces 1118 adapted to engage the proximally facing shield locking surfaces 1218 during operation. In the shown embodiment the skirt portion further comprises a pair of radially protruding opposed drop lock release flanges 1114 adapted to engage corresponding actuator leg release surfaces 1524 (see FIG. 27CX), a pair of radially protruding opposed torque flanges 1119 adapted to engage the shield torque ribs 1219, as well as a pair of opposed indicator cut-outs 1115. The torque interface could also be located on other portions of the shield and the hub, e.g. between the assembling arms and the hub tower portion.

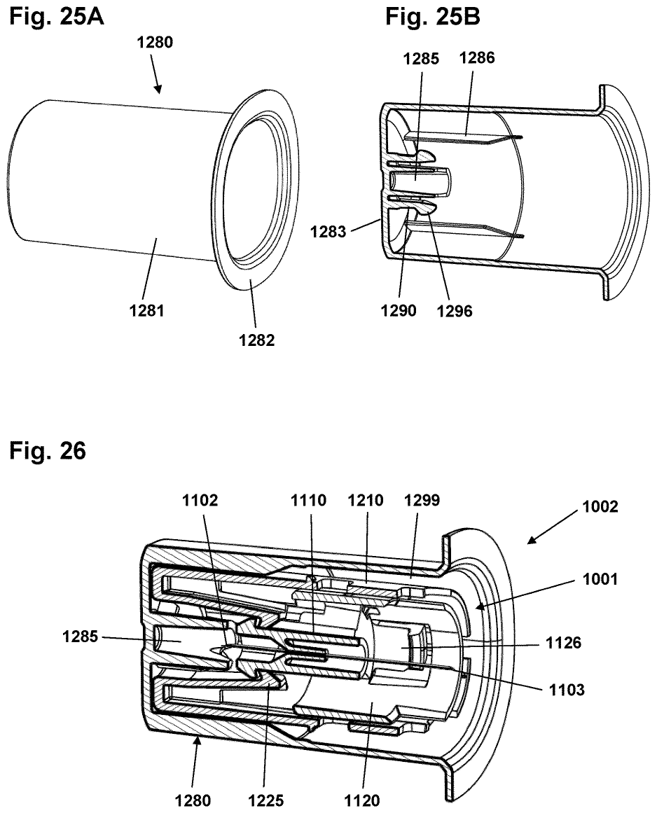

As shown in FIGS. 25A and 25B the container 1280 has a generally tubular configuration with a super-elliptic circumferential outer wall 1281 corresponding to the super-elliptic circumferential outer wall of the shield, a proximal opening with a circumferential flange 1282 and a closed distal end 1283 from which a tower structure 1285 and a pair of opposed snap lock fingers 1290 extend axially inwardly. The snap lock fingers each comprises an outwardly oriented snap protrusion 1296 adapted to releasably engage the shield tower grip edge portions 1216 to provide a snap coupling. Distally the container further comprises a plurality of inner support ribs 1286 adapted to engage the shield outer surface and support the shield when arranged in the container. With the shield mounted in the container the proximal portion provides a circumferential space 1299 (see FIG. 26) between the container and the shield allowing the 23 correspondingly shaped drug delivery housing portion to be received therein during mounting of the needle unit on the drug delivery device.

During assembling the hollow subcutaneous needle 1101 with bevelled proximal and distal ends is arranged in the hub bore and secured in place, e.g. by means of adhesive, this providing a free distal end portion 1102 and a free proximal end portion 1103. In the shown embodiment the needle proximal end portion extends proximally from the tower portion 1110 but does not extend proximally of the proximal-most portion of the hub 1100, this reducing the risk of accidental needle contact. The hub 1100 is subsequently inserted in the shield 200 with the stop surfaces 1121 and the ramp surfaces 1131 rotationally aligned with the shield stop surfaces 1221 respectively the shield ramp surfaces 1231, this allowing the shield stop surfaces to snap into engagement with the hub stop surfaces 1121. Also the hub coupling arms 1126 are rotationally aligned with the shield actuation ribs 1213. In the shown embodiment the hub proximal end is arranged slightly proximally of the shield proximal edge 1211. The assembled needle unit is then inserted into the container with the container snap lock fingers 1290 snapping into engagement with the shield tower grip edge portions 1216 to form a needle assembly. As seen in FIG. 26 an axial gap is provided between the container tower proximal end and the hub tower distal end. As a final assemble step a flexible foil member (not shown) is attached to the container proximal flange 1282 thereby sealing the interior for subsequent sterilization. FIG. 26 shows in cross-section the needle assembly 1002 with the needle unit 1001 positioned in the container 1280 before the seal foil is attached.

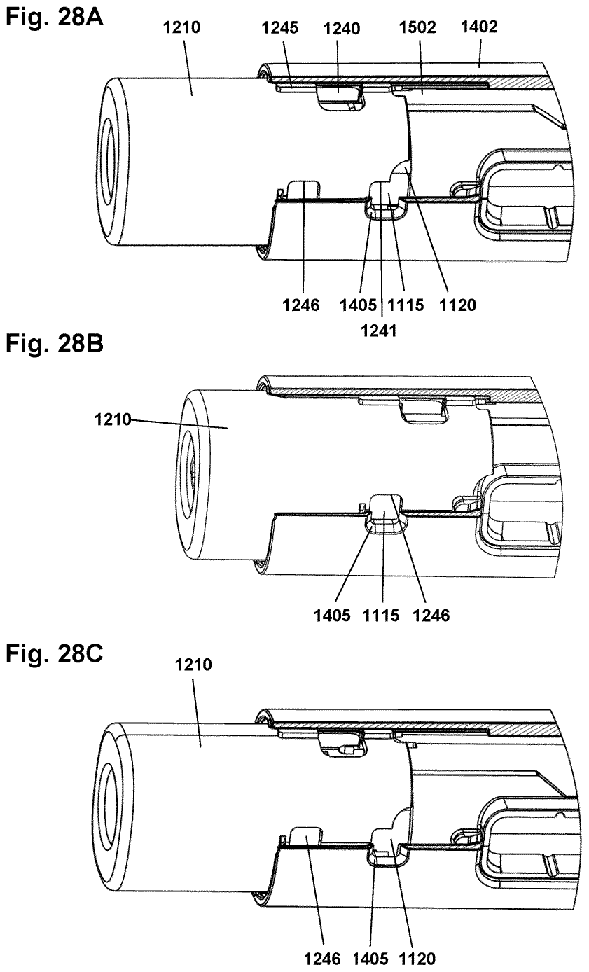

In the following the different features and aspects of the above-described needle unit and container combination will be described with reference to FIGS. 27A-27J showing a needle unit being mounted on a corresponding drug delivery device, operated to allow an amount of fluid drug to be injected subcutaneously and subsequently removed from the drug delivery device. The indicator functionality is additionally shown in FIGS. 28A-28C.

After the flexible seal foil has been removed from the container 1280 by the user, the container is intended to be used as a tool to mount the needle unit on the drug delivery device 1001 with a corresponding cartridge mount 1310, see FIG. 27A. In the shown embodiment the cartridge mount is arranged proximally of the distal end of the drug delivery device housing cartridge portion 1402 with a circumferential space 1403 between the cartridge holder 1300 and the housing adapted to receive the shield 1200 proximal portion in non-rotational engagement by the cooperating mounting ribs 1245 and shield slots 1415. In the shown embodiment the non-circular super-elliptic design of the container, the shield, the housing outer surface and the inner circumferential space makes it easy for the user to orient the needle assembly rotationally correct relative to the drug delivery device in either of its two possible rotational positions.

Alternatively, the non-circular configuration of the shield, container and housing may be a non-symmetrical form providing one rotational mounting position, an oval, elliptic, or rectangular form providing two rotational mounting positions, a triangular form providing three rotational mounting positions, or a square form providing four rotational mounting positions.

Initially the locked hub coupling arms 1126 engage the cartridge mount coupling flange portions 1311, this allowing the shield to be pushed forward by the container to an axial position in which the shield actuation ribs 1213 are not engaging the flexible hub arm outer surfaces 123, see FIG. 27B. The control arms 1230 are free to bend outwardly to allow proximal shield movement, but they will not snap over the ramp surfaces 1131. It is to be noted that in FIG. 27B the two structures for drawing reasons are shown as overlapping. Alternatively a clearance could be provided between the two structures.

The container assures that the shield and hub can be pushed firmly into engagement with the cartridge mount by the user, this allowing the free proximal needle end portion 1103 to penetrate the cartridge septum 1394 and the flexible hub coupling arms 1126 to be initially moved radially outwards in the receiving shield windows 1240 and subsequently snap radially inwards into engagement with the corresponding snap coupling flanges 1311 on the cartridge mount, see FIG. 27C.

During the axial coupling movement of the needle unit the hub drop lock release flanges 1114 engage the inclined leg release surfaces 1524 on the spring-biased actuator legs 1502. Initially the actuator is moved axially until the anti-rotational clutch portions 1517 are moved out of engagement with the housing, this allowing the actuator to be rotated by the axial movement of the hub. To counter the torque exerted on the hub during rotation of the actuator, the hub is supported by the shield (which is non-rotationally coupled to the housing 1400) via the torque flanges 1119 engaging the torque ribs 1219. Subsequently the shield actuator ribs 1213 engage the leg actuation surfaces 1523 of the actuator legs 1502 and moves axially together with the drop lock release flanges 1114. Depending on the actual design of the different components, the actuator may be fully rotated (here: 20 degrees) during mounting of the needle unit. Alternatively, final rotation of the actuator may take place when the actuator subsequently is allowed to be moved distally by the return spring 1590.

Axial mounting movement of the needle unit stops when the hub engages the cartridge mount, this indicating to the user that the needle unit has been mounted on the cartridge hub. When the user stops pushing on the container (or starts to pull the container away) the spring-biased actuator legs 1502 will push the shield 1200 slightly distally until the distally facing axial stop surfaces 1221 on the shield assembling arms engage the corresponding proximally facing stop surfaces 1121 on the hub tower portion. The control arms 1230 are moved back to their initial position. At the same time the clutch portions 1517 reengage with the housing lock ribs 1447 in the actuated rotational position. As the shield is moved distally the shield actuation ribs 1213 will move into blocking engagement with the flexible hub arm outer surfaces 1123 and thereby prevent radial outwards movement thereof, this securely locking the hub 1100 to the cartridge mount 1310 corresponding to an actuated hub coupling lock state in which a mounted needle unit cannot be removed from the drug delivery device, see FIG. 27D.

As the user pulls the container 1280 further distally to fully remove it, the container snap coupling 296 will disengage the shield 1200. When the container is fully removed the drug delivery device with the mounted needle unit 1001 is ready for use as shown in FIG. 27E. In this state the housing indicator opening 1405 is aligned with a shield first indicator opening 241 and a hub indicator cut-out 1115. The hub skirt 1120 is thus not visible to the user (see FIG. 28A).

When the user pushes the needle unit towards a skin surface the shield 1200 is pushed proximally allowing the needle distal end 1103 to be inserted subcutaneously. During initial proximal movement of the shield the ramp surfaces 1231 on the flexible control arms 1230 are pushed over the hub tower ramp surfaces 1131. As the shield is moved further proximally to its fully retracted position the shield actuator ribs 1213 pushes the pair of actuator legs 1502 proximally to thereby release the drug delivery device expelling mechanism thereby starting subcutaneous injection as described above, see FIG. 27F. As appears, the actuator legs 1502 serve both as a locking actuator for the hub coupling and as release members for the expelling mechanism. During out-dosing the shield is held in its fully retracted position by the snap lock 1135, 1235. In this state the housing indicator opening 1405 is aligned with the shield second indicator opening 246 and the hub indicator cut-out 1115. The hub skirt 1120 is thus not visible to the user (see FIG. 28B).

After a clicking sound generated by the expelling mechanism stops and the dose thus has been fully expelled, the user withdraws the needle unit from the skin surface thereby allowing the spring-biased actuator legs 1502 to push on the shield actuation ribs 1213 to thereby move the shield 1200 distally to its fully extended position again covering the needle distal portion 1103. During this movement the control surfaces 1232 on the control arms 1230 will engage the inclined proximally facing control surfaces 1132 on the hub tower (see FIG. 24A), which will force the needle hub 1100 to rotate as the shield is rotationally locked to the drug delivery device, see FIG. 27G. In the shown embodiment the hub is rotated 45 degrees relative to the shield, compare FIG. 27CX and 27GX.

During rotation of the hub relative to the shield a number of structures are moved into and out of engagement with each other:
(i) As the hub rotates, a portion of the hub skirt 1120 neighbouring the hub cut-out 1115 aligned with the housing indicator opening 1405 will rotate into alignment with the opening and thus be visible to the user (e.g. by having a contrasting colour), this indicating that the needle unit has been used and correspondingly that a dose of drug has been expelled. During drug expelling with the shield in its retracted position the shield second indicator opening 1246 is aligned with the housing indicator opening 405 (see FIG. 28C).
(ii) As the hub rotates, the flexible hub coupling arms 1126 rotate out of engagement with the shield actuation ribs 1213 thereby allowing the coupling arms to move radially outwards, see FIG. 27H, and thus the hub 1100 to be removed from the cartridge mount 1310. To reduce the force necessary to disengage the hub coupling arms from the cartridge mount, the hub coupling arms are rotated to a position on the cartridge mount having lower and less inclined release flange portions 1312.
(iii) As the needle is fixed in the hub it follows that the needle will rotate with the rotating hub, however, it may not be desirable that the needle rotates when fully inserted subcutaneously. Correspondingly, the hub and shield can be designed with an axial “play” before the shield control surfaces 1232 engages the inclined hub control surfaces 1132, this allowing the needle to be at least partly withdrawn from the skin before rotation starts. Indeed, the later rotation starts the steeper the inclination of the hub control surfaces have to be.
(iv) Before operation of the needle unit, the pair of locking ribs 1217 on the shield inner surface were free to move in the axial direction thus allowing the shield to be moved from its extended to its retracted position. As the hub is rotated the pair of distally facing locking surfaces 1118 are rotated into alignment with the proximal ends 1218 of the locking ribs 1217 thereby preventing repeated retraction of the shield and thus use of the needle unit, thereby providing a safety lock, see FIGS. 23B and 24A. Correspondingly, the locking surfaces should be designed to withstand relatively large forces to prevent re-activation of the needle unit, e.g. if the pen device is dropped on a hard surface or in a mis-use scenario. Further, in case the drug delivery device as in the present embodiment is shield released the shield lock will also serve as a double dose prevention means.
(v) In the shown embodiment the container is intended to be used as a tool also for removing the needle unit, see FIG. 27I. When the user re-attaches the container, the container snap coupling 1296 will engage the shield 1200. The container snap coupling is designed to have a release force larger than the release force necessary to pull the ridges 1127 of the flexible hub coupling arms axially out of engagement with the cartridge mount release flanges 1312, this allowing the needle unit to be removed from the cartridge mount securely held in the container after which the needle assembly 1002 can be safely discarded, see FIG. 27J. As the needle unit is locked to the container via the container snap coupling and the proximal end is positioned a certain distance inside the container and only being surrounded by a small free space, removal of the needle unit from the container would be difficult.
(vi) As the hub rotates, the drop lock release flanges 1114 are rotated out of alignment with the actuator leg release surfaces 1524, this preventing that a used and locked needle unit can be used to release the actuator drop lock.

### Alternative Embodiments

In the above-described embodiment, when the hub rotates relative to the cartridge mount the flexible coupling arms 1126 are rotated to align with the inclined release flanges 1312 allowing the hub coupling arms to disengage with ease which may encourage the user to remove the needle unit without using the container. To encourage the user to use the container, the release flanges may be modified to require a larger release force which would make it more difficult to merely grab and pull the shield out of engagement with the hub mount. To allow this the snap coupling between the container and the shield has to be able to transfer the required force, however, as the same snap coupling should be designed to allow for easy removal of the container after initial mounting of the needle unit this may not be desirable.

Correspondingly, a needle assembly may be provided comprising a snap coupling between the container and the shield, the snap coupling being actuatable between a first state in which the needle unit can be removed from the container using a first amount of force, and a second state in which the needle unit can be removed from the container using a second higher amount of force. The assembly may be operated between the two states by the rotational movement of the hub inside the shield. A detailed description of such an arrangement is described in co-pending application EP 23174814.6 which is hereby incorporated by reference.

In the above-described embodiment an indicator is incorporated in the drug delivery housing and being operated by the rotational movement of the hub. Although indicator actuation is controlled by the needle unit, the placement of the indicator window 1405 on the housing is designed to associate the indicator with operation of the device per se and thus indicate that a dose of drug has been expelled which will be the case when the shield has been returned to its extended and now locked position.

However, it may be desirable to provide the indicator on the shield to instead directly indicate that the needle module has been used and is now locked. Correspondingly, instead of the device housing the shield may be provided with an indicator window and the hub may be provided with an indicator surface which initially is not aligned with the window but moved into alignment therewith when the hub is rotated after use.

In the above description of exemplary embodiments, the different structures and means providing the described functionality for the different components have been described to a degree to which the concept of the present invention will be apparent to the skilled reader. The detailed construction and specification for the different components are considered the object of a nor-mal design procedure performed by the skilled person along the lines set out in the present specification.

1. A needle unit comprising:
a needle hub,
a hollow needle mounted in the needle hub and having a pointed distal end protruding from the needle hub, the hollow needle defining a reference axis,
a shield in which the needle hub is fully or partly arranged, the shield being axially moveable relative to the needle hub between an extended position in which the shield axially covers the needle distal end, and a retracted position in which the needle distal end protrudes from the shield corresponding to a needle free length,
a coupling allowing the needle unit to be used in combination with a drug delivery device, and
a linear-to-rotational movement converter mechanism causing the needle hub to be rotated relative to the shield when the shield is moved from its retracted to its extended position,
wherein the linear-to-rotational movement converter mechanism has an initial axial slack,
whereby rotational movement of the needle hub relative to the shield takes place after at least 25% of the needle free length has been covered by the shield.

2. The needle unit as in claim 1, wherein the linear-to-rotational movement converter mechanism comprises an inclined surface providing that axial movement of the shield relative to the needle hub is converted to rotation of the needle hub relative to the shield.

3. The needle unit as in claim 1, wherein rotational movement of the needle hub takes place after the needle free length has been covered 50%, 75% or 100% by the shield.

4. The needle unit as in claim 1, wherein:
when the shield is moved from the retracted to the extended position, the needle hub rotates relative to the shield to a rotational locking position in which the shield cannot be retracted.

5. The needle unit as in claim 1, wherein:
the coupling is a mounting coupling allowing the needle unit to be mounted on a drug delivery device.

6. The needle unit as in claim 5, wherein the mounting coupling comprises:
deflectable gripping fingers forming part of the needle hub and adapted to grip a corresponding coupling structure on the drug delivery device, and
blocking structure forming part of the shield and adapted to block radial movement of the gripping fingers, corresponding to a lock state.

7. The needle unit as in claim 6 in combination with a drug delivery device, the combination forming a drug delivery assembly, the drug delivery device comprising:
a housing comprising or adapted to comprise a drug-filled cartridge,
an actuatable expelling mechanism for expelling a pre-set or user-set amount of drug from the cartridge, and
coupling structure allowing the needle unit to be mounted on the drug delivery device, the shield being mounted non-rotatable relative to the drug delivery device.

8. The drug delivery assembly as in claim 7, wherein the mounting coupling is actuatable between:
a lock state in which a mounted needle unit cannot be removed from the drug delivery device, and
an actuated release state in which the needle unit can be removed from the drug delivery device,
wherein the mounting coupling is actuated from the lock state to the release state by rotational movement of the needle hub relative to the shield.

9. The drug delivery assembly as in claim 8, wherein the mounting coupling comprises:
deflectable gripping fingers forming part of the needle hub and adapted to grip a corresponding coupling structure on the drug delivery device, and
blocking structure forming part of the shield and adapted to block radial movement of the gripping fingers, corresponding to the lock state,
wherein the deflectable gripping fingers are rotated from a lock position to a release position in which radial movement of the gripping fingers is possible, this allowing the needle unit to be removed from the drug delivery device.

10. The drug delivery assembly comprising a needle unit as in claim 1, the drug delivery assembly further comprising:
a housing comprising or adapted to comprise a drug-filled cartridge,
an actuatable expelling mechanism for expelling a pre-set or user-set amount of drug from the cartridge, and
coupling structure allowing the needle hub to rotate relative to the housing and the shield to move axially and non-rotatable relative to the housing.

11. The drug delivery assembly as in claim 10, wherein the needle unit cannot be removed from the housing.
